(19) United States
(12) Patent Application Publication
(10) Pub. No.: US 2014/0135751 A1
(43) Pub. Date: May 15, 2014

(54) SYSTEM AND METHOD FOR INCISING A TILTED CRYSTALLINE LENS

(71) Applicants: Kristian Hohla, Muenchen (DE); Holger Schlueter, Muenchen (DE); Frieder Loesel, Mannheim (DE); David Haydn Mordaunt, Los Gatos, CA (US)

(72) Inventors: Kristian Hohla, Muenchen (DE); Holger Schlueter, Muenchen (DE); Frieder Loesel, Mannheim (DE); David Haydn Mordaunt, Los Gatos, CA (US)

(21) Appl. No.: 13/797,602

(22) Filed: Mar. 12, 2013

Related U.S. Application Data
(60) Provisional application No. 61/724,874, filed on Nov. 9, 2012.

(51) Int. Cl. A61F 9/008

(52) U.S. Cl. CPC A61F 9/008
USPC 606/6; 606/4

(57) ABSTRACT

A system and method are disclosed for using a laser unit to treat a crystalline lens (or lens capsule) to compensate for any tilt angle “φ” there may be between a lens axis and an operational axis of the laser unit (i.e. “z” axis). To begin, a contiguous sequence of procedure paths that collectively define the boundary surface of a lens volume are identified, with each procedure path inclined by the tilt angle “φ”. A slice occurs in an x-y plane that is on the boundary surface of the volume of lens and includes portions of several procedure paths. The slices are projected into the x-y plane where they are sequenced for use as trace paths for the laser unit. The trace paths are used to guide a laser beam to perform LIOB along the slice for the different values of “z” to incise the boundary surface.

This application claims the benefit of U.S. Provisional Patent Application Ser. No. 61/724,874, filed Nov. 9, 2012. The entire contents of Application Ser. No. 61/724,874 are hereby incorporated by reference herein.

### FIELD OF THE INVENTION

The present invention pertains generally to laser surgical procedures. More particularly, the present invention pertains to systems and methods for performing capsulotomy and lens fragmentation procedures. The present invention is particularly, but not exclusively, useful for performing capsulotomy and lens fragmentation procedures with a focused laser beam on a tilted crystalline lens.

### BACKGROUND OF THE INVENTION

In an ideal pre-surgical configuration, and with the patient in a supine position, the human crystalline lens is oriented horizontally. With this ideal configuration, a capsulotomy can be performed by creating circular incisions in a series of contiguous, horizontal planes. When a laser beam is used to create the incisions, the horizontal lens is generally orthogonal to the direction of beam propagation. However, for a typical patient, the lens may be tilted relative to an ideal horizontal configuration, and, as a consequence, may not necessarily be oriented orthogonal to the direction of laser beam propagation. In fact, the art describes how a human lens can appear tilted in the laser coordinate system. Generally, the tilt will be the result of docking an eye with a laser unit when the eye is not directed straight towards the laser unit. One way to measure the tilt of a lens consists in producing an image using optical coherence tomography (OCT) along the circular circumference of a planned capsulotomy, and ‘unfolding’ the three-dimensional (3-D) scan surface into two-dimensional (2-D). When the lens surface in this unfolded 2-D rendition has a sinusoidal shape, tilt is present.

One way to adjust a capsulotomy (or other circularly symmetric) pattern to compensate for lens tilt involves transferring the laser scan pattern into the coordinate system of the lens by tilting the pattern by the same angle as the eye/lens. Of course, when the laser coordinate system and the lens coordinate system are aligned (i.e. there is no lens tilt), no transformation is required. In the presence of tilt, a transformation that tilts the pattern by the same angle as the eye/lens (e.g. to produce a pattern consisting of tilted circles) requires the beam scanner to deflect the beam in “x”, “y”, and “z” directions during every rotation (i.e. for each circle). In general, scanning in “x”, “y”, and “z” directions is typically accomplished using raster graphics which are relatively slow and processor intensive. An alternative to raster graphics is vector graphics in which 2-D images can be represented and manipulated much more conveniently than using raster graphics. In particular, transformations using vector graphics are quicker and less processor-intensive than an approach in which each individual point of the procedure path is transformed using matrices.

In light of the above, it is an object of the present invention to provide systems and methods for performing capsulotomy and lens fragmentation procedures with a focused laser beam on a tilted crystalline lens. Another object of the present invention is to provide systems and methods for incising tissue on a procedure path that is calculated by a processor to accommodate for lens tilt that are relatively quick in terms of processor speed and are processor efficient. Yet another object of the present invention is to provide systems and methods for incising tissue while accommodating for lens tilt by using a transformation in which the pattern remains horizontal, and adjusts in shape only in order to compensate for tilt. Still another object of the present invention is to provide systems and methods for incising a tilted crystalline lens which are simple to implement and relatively cost effective.

In accordance with the present invention, a method for using a laser unit to perform Laser Induced Optical Breakdown (LIOB) on transparent material (e.g. tissue of a lens capsule, or the crystalline lens within the capsule) is provided to compensate for any tilt angle “φ” there may be between an optical axis of the transparent material and an operational axis of the laser unit. To begin, this methodology requires identifying a procedure path on a surface of the transparent material, relative to the optical axis of the material (e.g. lens capsule). A contiguous sequence of such procedure paths can then be identified which, collectively, will define the boundary surface of a volume of the transparent material.

As envisioned for the present invention, each procedure path in the sequence will be inclined by the tilt angle “φ” relative to the operational axis of the laser unit (i.e. a “z” axis). Consequently, a slice in an x-y plane that is on the boundary surface of the volume of material (tissue) will be perpendicular to the operational (“z”) axis. And, it will include portions of several procedure paths. In the event, these slices are effectively projected into the x-y plane where they are sequenced for use as trace paths for the laser unit. The same slicing and projecting technique is then repeatedly used, with each slice corresponding with a change in the “z” direction by a predetermined distance “Δz”. Thus, slices in a sequence of x-y planes are projected as trace paths for different values of “z”.

Functionally, the trace paths created as described above are used to guide a laser beam to perform LIOB along the slice for the different values of “z”. Once all values of “z” have been used, the entire boundary surface of the volume of tissue will have been altered by LIOB. As will be appreciated by the skilled artisan, additional LIOB can be performed within the boundary of the volume of tissue in order to accomplish a lens fragmentation procedure.

In an alternate embodiment of the present invention, the plurality of procedure paths described above can each be projected into an x-y plane. In this embodiment, each procedure path will be an actual path and, depending on the extent of the tilt angle “φ”, all of the resultant actual paths will have a same sinusoidal variation in “z”. At this point it is to be noted that this sinusoidal variation for one of the actual paths can be unfolded into a two-dimensional representation that is useful for determining the magnitude of the tilt angle “φ”.

As envisioned for the alternate embodiment of the present invention, each procedure path (actual path) can be projected into an x-y plane to create a respective trace path. The laser beam can then be guided along respective trace paths, and while using the appropriate sinusoidal variation in “z”, LIOB can be performed on the boundary surface of the volume of tissue with the same result mentioned above. In an operation, there will be an “n” number of laser beam changes, with each change being a move through a distance “d” between adjacent actual paths. This requires that a corresponding “n+1” number of trace paths be created. In detail, to create the sequence of trace paths, each successive trace path is established by moving from the immediately previous trace path through a distance Δxₙ=d sin φ in the x-y plane, and through a distance Δzₙ=d cos φ in the “z” direction from the previous x-y plane.

Structurally, the system for the present invention includes a laser unit for generating a laser beam. Importantly, the focal point of the laser beam must be capable of performing Laser Induced Optical Breakdown (LIOB). In this case, the laser unit defines an orthogonal x-y-z reference, and the tilt angle “φ” is determined between the defined axis (optical axis) of the material (lens tissue), and the “z” axis of the laser unit.

The system also includes a detector (e.g. an OCT imaging unit) for describing a surface for LIOB within the crystalline lens or lens capsule (i.e. a transparent material). As indicated above, the surface to be altered by LIOB is established relative to the defined axis of the transparent material (e.g. lens, lens capsule). Also, the detector is used to identify a slice on the surface of the material that is to be altered. Importantly, this slice will lie in an x-y plane that is defined by the laser unit, and it will have a unique z-value. As a practical matter, the slice will have a thickness of “Δz” that corresponds with depth of focus of the laser beam focal point.

A computer is connected with both the laser unit and the detector. In this combination, the computer uses each slice identified by the detector to electronically create a respective trace path for the laser unit. Once a trace path is identified, the computer then actuates the laser unit and guides its laser beam along the trace path to perform LIOB at a succession of laser beam focal points on the surface of the material. Further, as stated above, this is done while maintaining “z” constant. Changes in “z” (i.e. Δz) are also controlled by the computer.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the operational components for a system in accordance with the present invention;

FIG. 2 is a perspective view of a lens with a circular path on its surface;

FIG. 3 is a tilted version of the lens shown in FIG. 2;

FIG. 4 shows a profile view of a tilted lens illustrating x-y sub-patterns oriented on a laser unit oriented axis;

FIG. 5 shows a profile view of a tilted lens illustrating x-y sub-patterns oriented on a lens oriented axis;

FIG. 6 shows an x-y-z lensfrag envelope positioned between x-y incision planes;

FIG. 7 is a view of a lensfrag envelope as seen relative to x-y planes along the line 7-7 in FIG. 6.

FIG. 8 is a view of the lensfrag envelope shown in FIG. 7 with a greater tilt angle “φ”;

FIG. 9 shows arcuate incision lines in respective x-y planes;

FIGS. 10, 11, 12 and 13 are views of incision lines in a lensfrag envelope as seen along the line 7-7 in FIG. 6;

FIG. 14 is a presentation of interrelated geometric perspectives for an LIOB path as influenced by the tilt of a lens relative to a laser beam path;

FIG. 15 is a two-dimensional presentation of a circular path on a lens surface, for use in determining a tilt angle “φ”;

FIG. 16 shows the geometrical relationship between changes in “Δx” and “Δy” that result in a change of “Δz“; and

FIG. 17 illustrates the relationship between a change “Δz” on the trace path to be followed by a laser beam during an operation of the present invention.

### DESCRIPTION OF THE PREFERRED EMBODIMENTS

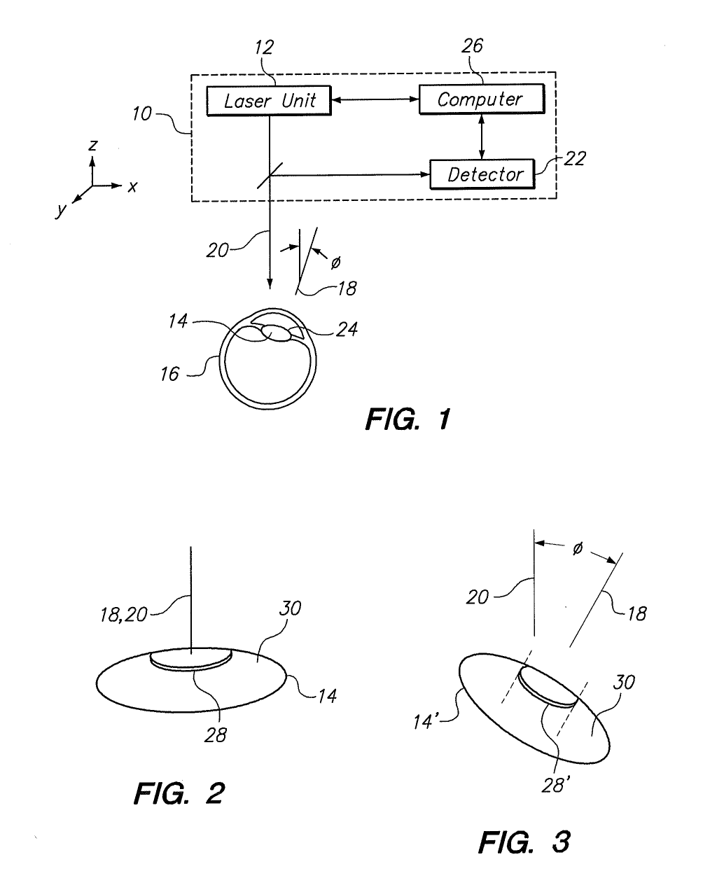

Referring initially to FIG. 1, a system 10 for using a laser unit 12 to perform Laser Induced Optical Breakdown (LIOB) on tissue of a lens 14 of an eye 16 is shown. As detailed below, the system 10 and the methods provided herein can be used to perform ocular procedures including, but not necessarily limited to, capsulotomy procedures and lens fragmentation procedures. Moreover, as illustrated in FIG. 1, these procedures can be performed while compensating for any tilt angle “φ” there may be between an optical axis 18 of the lens 14 and an operational axis 20 of the laser unit 12. For the system 10, the laser unit 12 is typically a so-called femtosecond laser generating a laser beam that can be focused to a subsurface location. Importantly, the focal point of the laser beam must be capable of performing Laser Induced Optical Breakdown (LIOB). In this case, the laser unit 12 defines an orthogonal x-y-z reference.

FIG. 1 further shows that the system 10 can include a detector 22 (e.g. an OCT imaging unit) for describing a surface for LIOB within the crystalline lens 14 or lens capsule 24. As indicated above, the surface to be altered by LIOB is established relative to the lens axis 18. Also, for the system 10, the detector 22 can be used to identify a slice (see more detailed description below) on the surface of the lens 14 or lens capsule 24 that is to be altered. Importantly, this slice will lie in an x-y plane that is defined by the laser unit 12, and it will have a unique z-value. As a practical matter, the slice will have a thickness of “Δz” that corresponds with depth of focus of the laser beam focal point.

Continuing with FIG. 1, a computer 26 is connected with both the laser unit 12 and the detector 22. In this combination, the computer 26 can use each slice identified by the detector 22 to electronically create a respective trace path for the laser unit 12. Once a trace path is identified, the computer 26 then actuates the laser unit 12 and guides its laser beam along the trace path to perform LIOB at a succession of laser beam focal points on the lens 14 or lens capsule 24. Further, as stated above, this is done while maintaining “z” constant. Changes in “z” (i.e. Δz) are also controlled by the computer 26.

FIG. 2 shows a lens with a circular incision 28 on the capsule surface 30 of a lens 14 that is not tilted relative to laser axis 20 (i.e. the laser axis 20 and lens axis 18 are aligned). FIG. 2 illustrates that the circular incision 28 can be performed by scanning the focal spot of a laser beam on circular paths in the x-y plane (see FIG. 1) while changing the depth or “z” parameter at the beginning of each path.

FIG. 3 illustrates a circular incision 28′ on a lens 14′ that is tilted (i.e. having a lens axis 18 that is oriented at a tilt angle “φ” to the operational axis 20 of the laser unit 12 (FIG. 1). FIG. 3 further illustrates that in the coordinate system of the laser unit 12 (FIG. 1), the circular incision 28′ becomes an elliptical shape in the x-y plane of the laser when tilt is present.

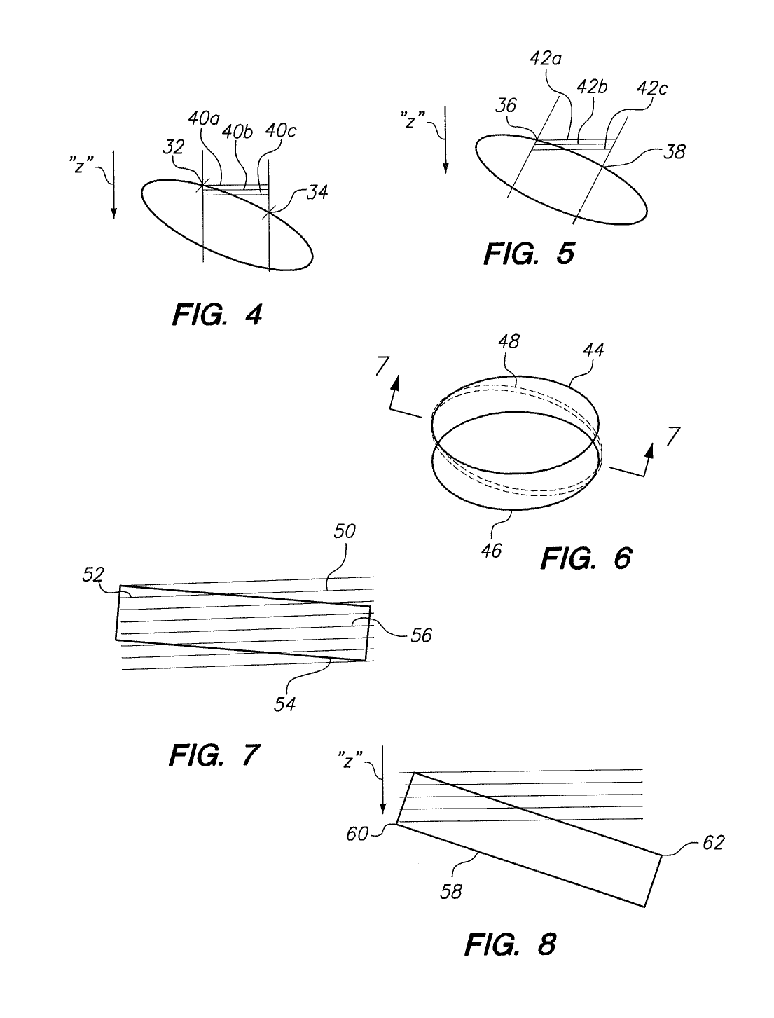

FIGS. 4 and 5 show two ways that x-y sub-patterns can be implemented. Specifically, FIG. 4 shows x-y sub-patterns 40a-c oriented on a laser unit oriented axis and FIG. 5 shows x-y sub-patterns 42a-c oriented on a lens oriented axis. Identified points 32, 34 on FIG. 4 and 36, 38 on FIG. 5 indicate the diameter of a planned capsulotomy. As shown in FIG. 4 and FIG. 5, the capsulotomy pattern is a series of x-y sub-patterns 40a-c (FIG. 4) and 42a-c (FIG. 5) that are repeated at successive depths “z” and visible as horizontal lines. While only a few such sub-patterns 40a-c (FIG. 4) and 42a-c (FIG. 5) are shown, in a typical procedure, the sub-patterns 40a-c (FIG. 4) and 42a-c (FIG. 5) will be closely spaced so as to create a contiguous cut along the capsulotomy. Instead of being the same circular shape as the desired capsulotomy, the projection of the outline of the circular capsulotomy into the x-y plane of the laser unit 12 (FIG. 1) is an ellipse where the ellipticity is determined by the degree of tilt. However, the change in the shape of the capsulotomy is minimal and is generally not clinically relevant. For example, for a capsulotomy of 5 mm diameter, an axial difference of about 0.8 μm is induced at 1 degree of tilt, an axial difference of about 6.9 μm is induced at 3 degrees of tilt, an axial difference of about 19 μm is induced at 5 degrees of tilt, and an axial difference of about 1.52 μm is induced at 10 degrees of tilt. For the two approaches illustrated by FIGS. 4 and 5, different calculations are used for movement in the “z” direction. It is also apparent from FIGS. 4 and 5 that the lengths of the horizontal incisions are different in each case. In the case of FIG. 4, the major axis (not shown) of an elliptical x-y incision (for a circular capsulotomy) lies perpendicular to the plane of the drawing. In FIG. 5, the major axis (not shown) of an elliptical x-y incision correspondingly lies (for a circular capsulotomy) in the plane of the drawing.

As shown in FIG. 6, the topmost x-y plane 44 and bottommost x-y plane 46 are necessary and yet intersect only very marginally with the capsule 48, which means that most of the time and energy expended on the topmost and bottommost rings is extraneous. It is to be appreciated that there is the desire to minimize the duration of the procedure and the radiation that enters the eye. Creating a capsulotomy in a tilted lens 14 (FIG. 1) using incisions in x-y planes therefore threatens to both lengthen the procedure and to increase the amount of laser radiation exposure that the eye 16 receives.

FIG. 7 illustrates that significant parts (fragments 50) of the sub-patterns 52 do not intersect with the lens fragmentation (lensfrag) envelope 54, resulting in time and energy being wasted on such line fragments 50. The solution consists of incising only those parts 56 that lie on and within the lensfrag envelope 54.

FIG. 8 illustrates that for a capsulotomy (envelope 58) that has a limited depth (z-range) and/or in the presence of significant tilt, point 60 lies higher than point 62. This has the consequence that certain x-y planes intersect with the capsulotomy envelope 58 on segments that are not connected with the minor axes (not shown) of the ellipses (for the case when the x-y sub-patterns are oriented on a laser unit axis (FIG. 4) or the major axis (not shown) when the x-y sub-patterns are oriented on a lens oriented axis (FIG. 5).

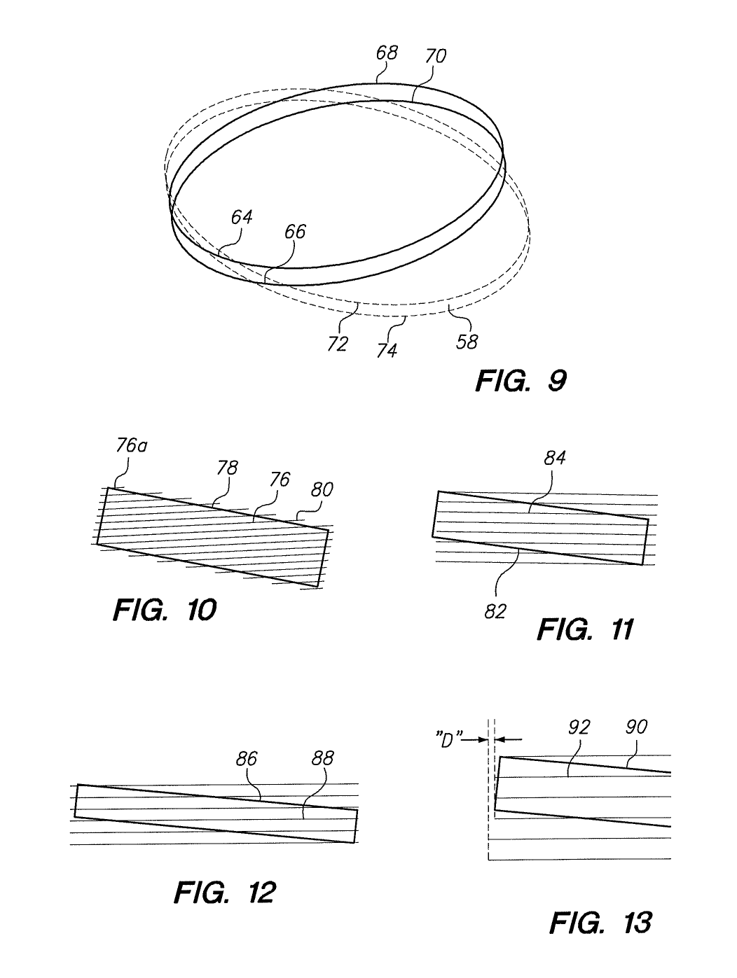

FIG. 9 shows arcuate incision lines 64, 66 in respective x-y planes 68, 70 for the lens tilt and the “z” direction shown in FIG. 8, showing the capsulotomy envelope 58 having a top end 72 and a bottom end 74.

FIG. 10 shows sub-patterns 76 for an envelope 78 when the situation shown in FIGS. 8 and 9 does not arise, showing that an extra margin 80 can be added in length in order to be sure that each incision intersects with the capsulotomy envelope 78 without which the capsulotomy incisions as a whole would not be complete. Another type of extra margin can be created that takes the form of additional x-y incisions (not shown) above the topmost line sub-pattern 76a shown in FIG. 10. While these additional incisions would not intersect with the capsulotomy envelope 78, they would provide further assurance that the capsulotomy will be complete by compensating for slight inaccuracies in the measurement of lens tilt and/or expanding the tolerance imposed on the focal spot position, for example.

FIG. 11 shows a profile view of a lensfrag envelope 82 (solid rectangle) viewed along the axis of tilt with superimposed exemplary x-y sub-patterns 84 along which the laser unit 12 (FIG. 1) can perform incisions. While a lensfrag pattern may be one of many different shapes, assume for the present purpose that it is comprised of horizontal planar incisions. When, as in the case, the pattern consists of a series of identically shaped ellipses that are displaced only in “z” direction with respect to each other, the outlines of the ellipses do not generally coincide with the lensfrag envelope 82. In FIG. 8 only one such line corresponds to the lensfrag envelope 58, with the result that the minor axis (not shown) of the ellipse (which lies in the plane of the drawing) needs to be modulated in order for the minor axis of the x-y incisions to match the outline of the desired lens fragmentation and not enter a safety zone which protects the lens capsule 24 (FIG. 1) from LIOB during lens fragmentation. The laser incisional patterns of FIG. 5, in contrast, do not need to vary in diameter, and are therefore conceptually simpler to implement as shown in FIGS. 12 and 13.

FIG. 12 shows another example of a lensfrag envelope 86 (solid rectangle) viewed along the axis of tilt with superimposed exemplary x-y sub-patterns 88 along which the laser unit 12 (FIG. 1) can perform incisions.

FIG. 13 shows yet another example of a lensfrag envelope 90 (solid rectangle) viewed along the axis of tilt with superimposed exemplary x-y sub-patterns 92 along which the laser unit 12 (FIG. 1) can perform incisions. FIG. 13 illustrates that with every additional plane that is incised, the identically shaped ellipses are displaced by a certain amount, “D” along the direction of tilt (see FIG. 5). With this displacement, the outline of the ellipses will lie on and within the lensfrag envelope 90. It is to be appreciated that instead of stacked circles or ellipses, the lensfrag envelope 90 may equally well be a (circular or elliptical) spiral (not shown).

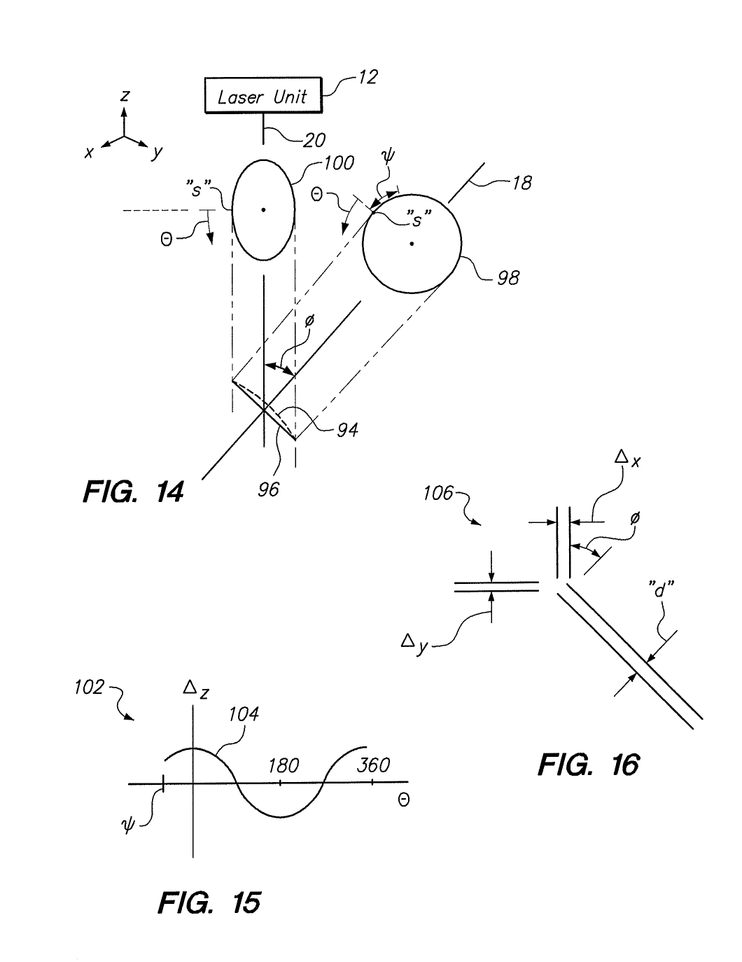

FIG. 14 is a presentation of interrelated geometric perspectives for an LIOB path as influenced by the tilt of a lens relative to a laser beam path. As shown, actual surface 94 of a transparent material, such as the lens 14 shown in FIG. 1) is tilted such that an angle “φ” is established between an optical axis 18 of the transparent material and an operational axis 20 of the laser unit 12. An actual procedure path 96 on a surface 94 of the transparent material, relative to the optical axis 18 of the material (e.g. lens capsule) can be identified. A contiguous sequence of such actual procedure paths 96 can then be identified which, collectively, will define the boundary surface of a volume of the transparent material. As shown, the actual procedure path 96 in the sequence will be inclined by the tilt angle “φ” relative to the operational axis 20 of the laser unit 12 (i.e. a “z” axis). In addition, as shown, a projected procedure path 98 of the actual procedure path 96, taken along the optical axis 18, can be identified, which for the case presented is substantially circular.

Continuing with FIG. 14, it can be seen that a trace path projection 100 of the actual procedure path 96, taken along the operational axis 20, can be identified, which for the case presented is substantially elliptical. During a procedure, FIG. 14 indicates that the trace path projection 100 can be used to direct a laser beam from the laser unit 12 to perform LIOB on the transparent material beginning at a start point “S” and continuing around the elliptical trace path projection 100 (i.e. through rotation angle “θ”).

FIG. 15 shows a 2-D rendition 102 that illustrates that the tilt of a lens 14 (FIG. 1) can be measured by producing an image using optical coherence tomography (OCT) along the circular circumference (i.e. through 360 degrees) of a planned capsulotomy, and ‘unfolding’ the 3-D scan surface into the 2-D rendition 102. When the lens surface is unfolded, the 2-D rendition 102 has a sinusoidal shape 104 when tilt is present. More specifically, the tilt of a lens 14 (FIG. 1) can be measured by creating a three-dimensional (3-D) image of an orienting path on a surface of the capsule, wherein the orienting path is a circle centered on the optical axis of the lens capsule; unfolding the image of the orienting path into a two-dimensional graph to determine variations of the orienting path in a “z” direction relative to the x-y plane; and using the variations of “z” direction in the two-dimensional graph of the orienting path to determine a tilt angle “φ” of the optical axis 18 relative to the operational axis 20. In this process, as indicated in FIGS. 14 and 15, a correction angle “Ψ” for locating a start point, “s”, on the orienting path can be determined. In particular, the correction angle “Ψ” locates the start point at a maximum variation from the orienting path in the “z” direction from the x-y plane.

FIG. 16 shows the geometrical relationship (generally designated 106) between changes in “Δx” and “Δy” that result in a change of “Δz”. For the methods described herein, the trace path projection 100 created as described above with reference to FIG. 14 is used to guide a laser beam to perform LIOB along the slice for the different values of “z”. Once all values of “z” have been used, the entire boundary surface of the volume of tissue will have been altered by LIOB. As shown in FIG. 16, to create the sequence of trace paths, each successive trace path is established by moving from the immediately previous trace path through a distance Δxₙ=d sin φ in the x-y plane, and through a distance Δzₙ=d cos φ in the “z” direction from the previous x-y plane.

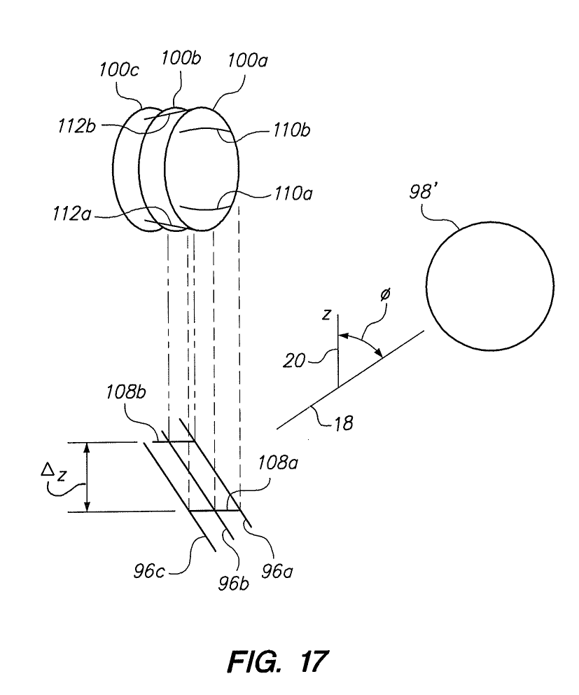

FIG. 17 illustrates the relationship between a change “Δz” on the trace path to be followed by a laser beam during an operation of the present invention. As shown, three actual procedure paths 96a-c correspond to a circular projection 98′ of the actual procedure paths 96a-c, taken along the optical axis 18, and respective elliptical trace path projection 100a-c of the actual procedure paths 96a-c, taken along the operational axis 20. As shown, each actual procedure path 96a-c in the sequence will be inclined by the tilt angle “φ” relative to the operational axis 20 (i.e. a “z” axis). Consequently, a slice 108a in an x-y plane that is on the boundary surface of the volume of material (tissue) will be perpendicular to the operational (“z”) axis. And, it will include portions of several procedure paths 96a-c. Specifically, as shown, slice 108a corresponds to trace paths 110a,b and slice 108b corresponds to trace paths 112a,b, with slice 108a distanced from slice 108b by the distance “Δz”. In the event, the slices 108a,b are effectively projected into the x-y plane where they are sequenced for use as trace paths for the laser unit 12 (FIG. 1). The same slicing and projecting technique is then repeatedly used, with each slice corresponding with a change in the “z” direction by a predetermined distance “Δz”. Thus, slices in a sequence of x-y planes are projected as trace paths for different values of “z”.

While the particular System and Method for Incising a Tilted Crystalline

Lens as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for performing Laser Induced Optical Breakdown (LIOB) with a laser unit on tissue of a lens capsule, wherein there is a tilt angle “φ” between an optical axis of the lens capsule and an operational axis of the laser unit, the method comprising the steps of:
identifying a procedure path on a surface of the lens capsule;
projecting the procedure path to create a trace path on an x-y plane, wherein the x-y plane is perpendicular to the operational axis of the laser unit; and
using the trace path for directing a laser beam from the laser unit to perform LIOB on tissue of the lens capsule.

2. A method as recited in claim 1 further comprising the step of repeating the using step an “n” number of times to cause LIOB along a succession of “n+1” actual paths in the tissue of the lens capsule with a distance “d” between adjacent actual paths, wherein each successive trace path is moved a distance Δxₙ=d sin φ in the x-y plane, and wherein each corresponding successive actual path is moved a distance Δzₙ=d cos φ in a “z” direction from the x-y plane.

3. A method as recited in claim 1 wherein the procedure path is a circle and the trace path is an ellipse.

4. A method as recited in claim 1 further comprising the steps of:
creating a three-dimensional (3-D) image of an orienting path on a surface of the capsule, wherein the orienting path is a circle centered on the optical axis of the lens capsule;
unfolding the image of the orienting path into a two-dimensional graph to determine variations of the orienting path in a “z” direction relative to the x-y plane; and
using the variations of “z” direction in the two-dimensional graph of the orienting path to determine a tilt angle “φ” of the optical axis relative to the operational axis.

5. A method as recited in claim 4 wherein the using step determines a correction angle “Ψ” for locating a start point on the orienting path.

6. A method as recited in claim 5 wherein the correction angle “Ψ” locates the start point at a maximum variation from the orienting path in the “z” direction from the x-y plane.

7. A method as recited in claim 1 further comprising the step of defining a volume of tissue, wherein the volume of tissue is bounded by the procedure path in the identifying step, and wherein the using step is performed through the defined volume of tissue to effect a lens fragmentation procedure.

8. A computer program product for use with a computer for performing a laser capsulotomy on a lens capsule, wherein the computer program product comprises program sections for respectively: establishing an operational axis between a laser unit and the capsule; selecting an optical axis for the capsule, wherein the optical axis is substantially perpendicular to a surface of the capsule; identifying a procedure path for performing Laser Induced Optical Breakdown (LIOB) on tissue of the lens capsule, wherein the procedure path is centered on the optical axis; projecting the procedure path along the operational axis and onto the x-y plane to fix a trace path in the x-y plane for operation of the laser unit; and performing LIOB on the capsule along the procedure path by moving a laser beam from the laser unit along the trace path in the x-y plane.

9. A computer program product as recited in claim 8 wherein the procedure path is a circle and the trace path is an ellipse.

10. A computer program product as recited in claim 8 further comprising program sections for respectively: creating a three-dimensional (3-D) image of an orienting path on a surface of the capsule, wherein the orienting path is a circle centered on the optical axis of the capsule and wherein the image is a projection of the orienting path onto an x-y plane oriented perpendicular to the operational axis; unfolding the image of the orienting path into a two-dimensional graph to determine variations of the orienting path in a “z” direction relative to the x-y plane; and using the variations of “z” direction in the two-dimensional graph of the orienting path to determine a tilt angle “φ” of the optical axis relative to the operational axis, and to determine a correction angle “Ψ” for locating a start point on the orienting path.

11. A computer program product as recited in claim 10 wherein the correction angle “Ψ” locates the start point at a maximum variation from the orienting path in the “z” direction from the x-y plane.

12. A computer program product as recited in claim 10 further comprising a program section for defining a volume of tissue, wherein the volume of tissue is bounded by the procedure path.

13. A computer program product as recited in claim 11 wherein the defined volume of tissue is defined to effect a lens fragmentation procedure.

14. A computer program product as recited in claim 10 wherein the program section for performing LIOB is repeated an “n” number of times to cause LIOB along a succession of “n+1” actual paths in the tissue of the lens capsule with a distance “d” between adjacent actual paths, wherein each successive trace path is moved a distance Δxₙ=d sin φ in the x-y plane, and wherein each corresponding successive actual path is moved a distance Δzₙ=d cos φ in a “z” direction from the x-y plane.

15. A system for performing Laser Induced Optical Breakdown (LIOB) on a transparent material which comprises:
a laser unit wherein the laser unit defines an orthogonal x-y-z reference, and wherein there is a tilt angle “φ” between a defined axis of the material and the “z” axis of the laser unit;
a detector for describing a surface for LIOB within the transparent material, wherein the surface is established relative to the defined axis of the material, and for identifying a slice of the surface, wherein the slice lies in an x-y plane defined by the laser unit with a selected z-value, and wherein the slice has a thickness of “Δz”; and
a computer for using the slice to create a trace path for the laser unit, for activating the laser unit to perform LIOB at a succession of laser beam focal points on the surface of the material by directing the laser beam along the trace path while maintaining “z” constant, and for controlling the laser unit to change the location of the slice by an increment “Δz” to perform LIOB over the entire surface.

16. A system as recited in claim 15 wherein “Δz” is equal to the depth of focus of the laser beam focal point.

17. A system as recited in claim 15 wherein the laser unit comprises a femtosecond laser unit.

18. A system as recited in claim 15 wherein the detector comprises an optical coherent tomography detector.

19. A system as recited in claim 15 wherein the trace path is elliptical.

20. A system as recited in claim 15 wherein the transparent material is a crystalline lens.
